(19) United States
(12) Patent Application Publication
(10) Pub. No.: US 2023/0277270 A1
(43) Pub. Date: Sep. 7, 2023

(54) NEUROMUSCULAR CHARACTERISATION METHOD AND ASSOCIATED MEASURING BENCH

(71) Applicants: UNIVERSITE D'AIX-MARSEILLE, MARSEILLE CEDEX 7 (FR); CENTRE NATIONAL DE LA RECHERCHE SCIENTIFIQUE, PARIS (FR)

(72) Inventors: Arnaud HAYS, AUBAGNE (FR); André JACQUES, MARSEILLE (FR)

(21) Appl. No.: 18/020,045

(22) PCT Filed: Oct. 27, 2021
(86) PCT No.: PCT/EP2021/079774

§ 371 Date: Feb. 6, 2023

(30) Foreign Application Priority Data
Nov. 2, 2020 (FR) 2011204

(51) Int. Cl. A61B 90/00; A63B 21/00

(52) U.S. Cl. CPC A61B 90/06; A63B 21/4034; A61B 2090/064; A63B 2225/09

(57) ABSTRACT

A characterization method for the neuromuscular characterization of at least one lower and/or upper limb of an individual using a measurement bench is provided. This method comprises a step of placing the individual in the measurement bench, a step of unlocking the seat of the individual, a step wherein the individual lands on a force platform, a step wherein the individual pushes against the force platform, a step of acquiring signals and a step of processing data from the acquired signals. In a step, the carriage is damped and the seat is then blocked in a step at the end of its travel after the pushing step and is then returned gently to a test position.

### CROSS-REFERENCE TO RELATED APPLICATIONS

This application is a National Stage of International patent application PCT/EP2021/079774, filed on Oct. 27, 2021, which claims priority to foreign French patent application No. FR 2011204, filed on Nov. 2, 2020, the disclosures of which are incorporated by reference in their entirety.

### FIELD OF THE INVENTION

The present invention relates to a characterization method for the motive and neuromuscular characterization of at least one lower and/or upper limb of an individual, and to a measurement bench for implementing this method.

### BACKGROUND

The neuromuscular characterization of a lower limb of an individual is well known to those skilled in the art. Such characterization is for example disclosed in the document “Isocinetisme, Guide clinique, 2009, by M. Elio Di Palma” accessible via the following address: https://elitemedicale.fr/media/documentations/Easytech/isocinetisme/lsocinetique_ea sytech_guide_clinique.pdf. Isokinetic systems are used for kinaesthetic diagnosis, functional assessment, rehabilitation and training in cases of neuromuscular deficiency. They make it possible to gather objective information allowing a fact-based rehabilitation program to be created. Rehabilitation based on the monitoring of performance with metered muscular workload is not only a highly effective treatment for correcting muscular deficiency, but also a method that makes it possible to increase the ability to coordinate, which is something that is difficult to improve using conventional training methods.

An isokinetic exercise consists in a constant-speed movement in which the resistance automatically adapts itself to the force applied, provided that the speed of the movement is maintained. This type of movement ensures maximum muscle contraction throughout the entire exercise, and for each range of movement of a joint. The use of suitable instruments allows the exercises to be customized according to the age, gender and pathology and to define the best method for training/rehabilitating the muscle structures at each phase of the training or rehabilitation plan. In order to perform these isokinetic exercises, the individual is seated on a static seat and, using the forward part of one of their tibias, pushes against an automated force arm. Such isokinetic exercises are notably described in the thesis: “Développement d'une méthode d'exploration de la balance musculaire basée sur la modélisation du signal isocinétique [Development of a method for exploring muscle balance based on the modeling of the isokinetic signal]” by Maryne Cozette, which can be consulted at www.theses.fr/2019AMIE0018. Although the isokinetic tests are fairly simple to perform, they are accompanied by a certain number of disadvantages. For example, these tests are not very reliable and medical practitioners are unable to be fully reliant on the measurements taken in order to establish a full protocol for the resumption of physical activity following a musculo-articular injury. In addition, these tests measure only the forces of the joints at constant speed, muscle by muscle. Furthermore, for technical and safety reasons they are not suitable for the entire general public. Finally, measurements that are repeatable are difficult to obtain.

Document US2019/0282848 describes a leg press exercise machine. This exercise machine comprises:
a main frame,
a first rotating bar connected to a first end of the main frame,
a second rotating bar connected to a second end of the main frame,
a support assembly connected to each of the first and second rotating bars, where the rotation of the first and second rotating bars causes the support assembly to move with respect to the main frame;
a seat support bar;
a seat mounted on the seat support bar, and in which the support assembly keeps said seat support bar at a certain angle.

In this press exercise machine, the individual builds up their muscles by moving the seat on the seat support bar by extending their lower limbs by pressing against a footplate platform. The difficulty in moving the seat, and therefore the effort to be supplied by the individual, can be adjusted using a system of weights via a cable. During the course of the movement, the individual's feet are always in contact with the platform. The seat therefore moves freely on the seat support. This press exercise machine does not, however, constitute a measurement bench for implementing a neuromuscular characterization method. Furthermore, it does not allow maximum pushing thrust of the ballistic type under no load or light load, because the seat travel is insufficient and there is no associated braking.

There is therefore a need to provide a new neuromuscular characterization method that is reliable and easy to implement.

### SUMMARY OF THE INVENTION

The present invention seeks to at least partially meet this need.

More particularly, the present invention seeks to improve the detection of musculo-skeletal-articular risks in individuals presenting with a functional imbalance such as a muscular, proprioceptive, psychological or other form of imbalance.

A first subject of the invention is a characterization method for the neuromuscular characterization of at least one lower and/or upper limb of an individual using a measurement bench. This characterization method comprises:
a step of placing the individual on a seat of the measurement bench;
a step of moving the seat into a test position, said test position being farther back than a standard position in which the individual is in contact with a force platform via the at least one limb of the individual, said force platform being intended to measure a force generated by said at least one limb of the individual;
a step of locking the seat in the test position;
a step of loading the seat with a predetermined load;
a step of unlocking the seat, said seat being driven toward the force platform by the predetermined load;
a step in which the individual lands on the force platform;
a step in which the individual pushes against the force platform;
a step of acquiring signals regarding the force generated by the individual on said force platform during the landing step and/or the pushing step;
a step of processing data from said acquired signals.

The characterization method uses a movement of the individual. The individual is drawn toward the platform by the predetermined load. The measurement of the neuromuscular imbalances is therefore more precise because it conforms to the so-called “ballistic” human motion by making it possible to produce a first phase of maximum deceleration for a given load. Specifically, the effort generated by the individual on the platform is an eccentric effort similar to landing or to descending a staircase. A second, pushing, phase, which is also ballistic, makes it possible to produce a phase of maximum acceleration. During the course of the second phase, a concentric effort, similar to a jump of the “drop jump” type, is produced.

In one particular embodiment, the characterization method comprises a step of damping the seat after the step of pushing by the individual.

In one particular embodiment, the seat is returned, after the step of pushing by the individual, to the test position or to a removal position for removing the individual from the measurement bench. The seat can thus be blocked at the end of travel after the pushing step. It is then returned gently to the test position or to the removal position. That allows the individual to push without apprehension and without having to question the damping or the return of the carriage, as this is done automatically. The individual can therefore focus on their pushing task.

In one particular embodiment, the speed of movement of the seat toward the test position and/or toward the removal position is controlled.

In one particular embodiment, the speed of movement of the seat is controlled by a carriage anchored to said seat.

In one particular embodiment, the mobile carriage is unanchored from the seat when said seat is locked in the test position.

In one particular embodiment, prior to the step of unlocking the seat, the position of the force platform is adjusted on part of a frame of the measurement bench to suit the individual.

In one particular embodiment, the step of acquiring the signals involves the digitizing of said signals.

In one particular embodiment, the data processing step is performed afterward.

Another subject of the invention is a measurement bench for the neuromuscular characterization of at least one lower and/or upper limb of an individual. The measurement bench comprises:
a frame intended to rest on the floor;
a force platform intended to measure a force generated by the at least one limb of the individual;
a seat intended to receive the individual, said seat being designed to be able to move with respect to the frame as far as a test position, said test position being farther back than a standard position in which the individual is in contact with the force platform via the at least one limb of the individual;
a locking/unlocking device suitable for locking/unlocking the seat in the test position, said seat being driven toward the force platform by a predetermined load upon unlocking;
a loading device for loading the seat with the predetermined load;
an acquisition device acquiring information regarding the force generated by the individual on the force platform when said individual lands on and/or pushes against said force platform, said acquisition device comprising signal acquisition software; processing software for processing data from the acquired signals.

The measurement bench is intended to make it possible to measure forces generated, in one, two or three dimensions, by the lower limbs such as the legs and/or upper limbs such as the arms, and the speed attained by the human body propelled by these forces. This system is an integrated system able to manage measurements, the positioning of the subject, sensors, movement safety features, and do so in an automatically and integrated manner. The automated systems, both pneumatic and electrical, are managed by an industrial-grade digital controller suitable for interacting with the actuators of the measurement bench and with the data acquisition system. The measurement bench is produced in such a way as to make it possible to characterize, in all dimensions, the individual and their physical performance in complete safety, with no prior learning, and on any individual, whether sporting, non-sporting, healthy, injured or in the process of rehabilitation.

In one particular embodiment, the force platform is suitable for measuring said force generated by the individual in three mutually perpendicular directions and for measuring three moments about said perpendicular directions.

In one particular embodiment, the force platform is in at least two parts.

In one particular embodiment, the force platform comprises sensors of the piezoelectric type suitable for generating a signal having a passband at least equal to 1000 Hz.

In one particular embodiment, the measurement bench comprises a carriage and a locking bolt, said locking bolt being able to adopt a deployed position in which the locking bolt mechanically connects the carriage to the seat and a retracted position in which the locking bolt does not connect said carriage to said seat.

In one particular embodiment, the measurement bench comprises a toothed belt suitable for moving the carriage and a three phase motor suitable for actuating said toothed belt, the speed of movement of the carriage being controlled by a speed sensor.

In one particular embodiment, the loading device comprises an indexer and a stack of a plurality of weights, said indexer being suitable for inserting a pin into the stack so as to select the predetermined load.

In one particular embodiment, the measurement bench comprises a damping device to damp the seat after a push from the individual.

In one particular embodiment, the processing software operates afterward.

### BRIEF DESCRIPTION OF THE DRAWINGS

The present invention will be better understood from reading the detailed description of some embodiments considered by way of nonlimiting example and illustrated by the attached drawings in which:

FIG. 1 is a partial perspective view of a measurement bench according to the invention;

FIG. 2 is a side view of the measurement bench of FIG. 1;

FIG. 3 is a rear view of part of the frame of the measurement bench of FIGS. 1 and 2;

FIG. 4 is a rear view of another part of the frame of the measurement bench of FIGS. 1 and 2;

FIG. 5 is a front perspective view of a seat of the measurement bench of FIGS. 1 to 4;

FIG. 6 is a rear perspective view of the seat of the measurement bench of FIGS. 1 to 4;

FIG. 7 is an enlarged view of part of the frame of the measurement bench of FIGS. 1 and 2 centered on a motion-inducing device of said measurement bench;

FIG. 8 is a partial perspective view of a loading device of the measurement bench of FIGS. 1 and 2;

FIG. 9 is a partial perspective view of part of the frame of the measurement bench of FIGS. 1 and 2 which is situated beneath the seat of FIGS. 5 and 6;

FIG. 10 is a rear view of the measurement bench of FIGS. 1 and 2, centered on a mobile-mass management system of said bench;

FIG. 11 is an enlarged view of part of the mobile-mass management system of FIG. 10;

FIG. 12 illustrates the various steps of a characterization method for the neuromuscular characterization of at least one limb of an individual using the measurement bench of FIGS. 1 to 11.

### DETAILED DESCRIPTION

The invention is not restricted to the embodiments or variants presented, and other embodiments and variants will be clearly apparent to a person skilled in the art.

In the various figures, elements that are identical or similar bear the same references.

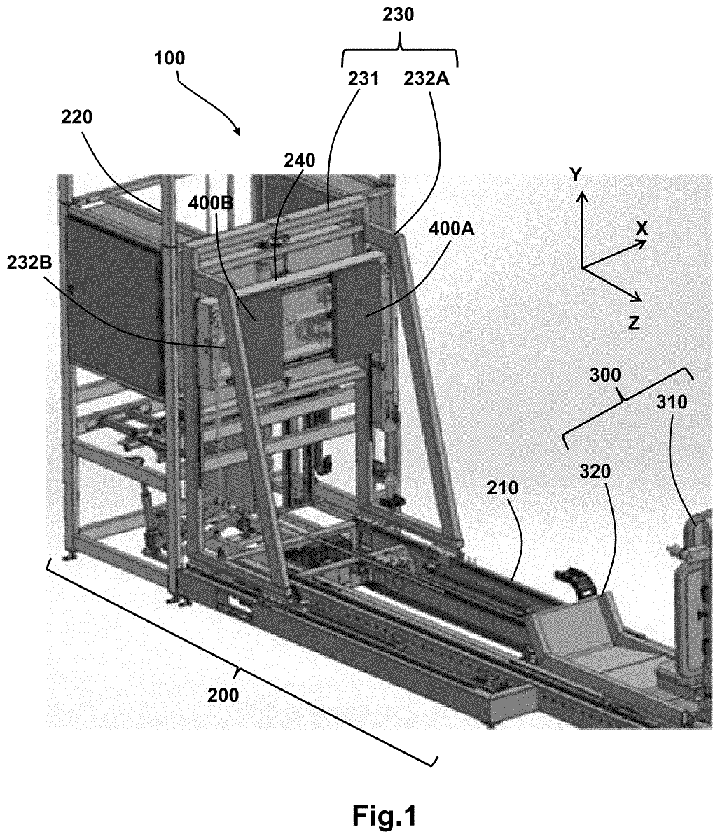

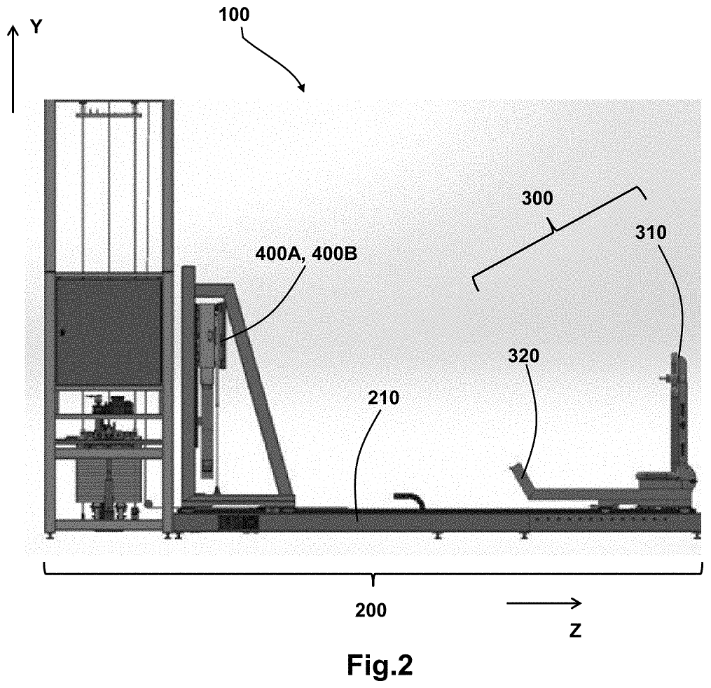

FIG. 1 and FIG. 2 schematically depict a partial perspective view of a measurement bench 100 according to the invention. This measurement bench 100 comprises:
a frame 200 intended to rest on the floor;
a seat 300 intended to receive the individual;
a force platform 400A, 400B intended to measure a force generated by the at least one limb of the individual;
a motion-inducing device 500 causing the seat 300 to move toward the force platform 400A, 400B, said motion-inducing device 500 being able to bring the seat 300 at a predetermined speed to a test position;
a device 600 for loading the seat;
a damping device 700;
a signal acquisition device comprising acquisition software;
processing software.

The frame 200 comprises:
a frame first part 210;
a frame second part 220;
a frame third part 230;
a frame fourth part 240.

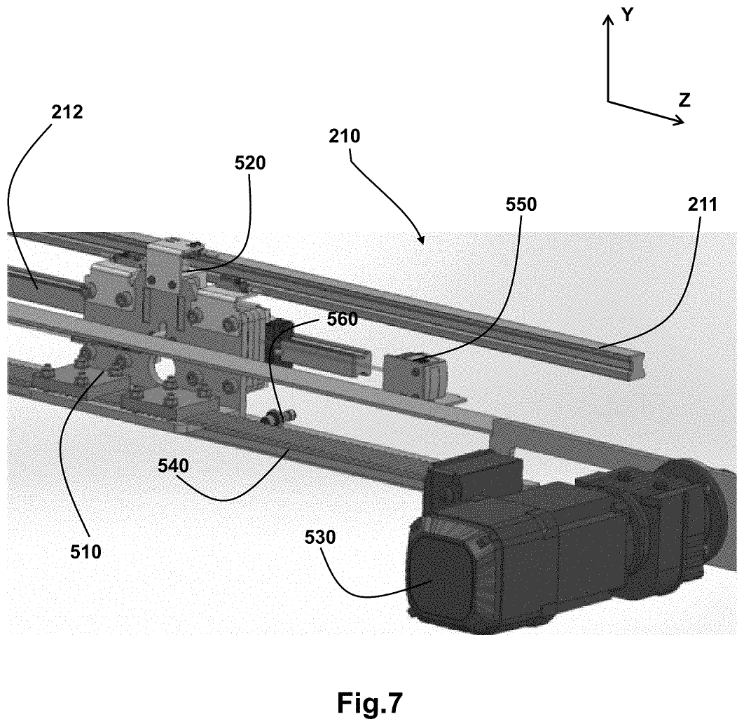

The frame first part 210 rests horizontally on the floor. As illustrated in FIG. 7, this frame first part 210 comprises:
a first rail 211;
a second rail 212 parallel to the first rail 211.

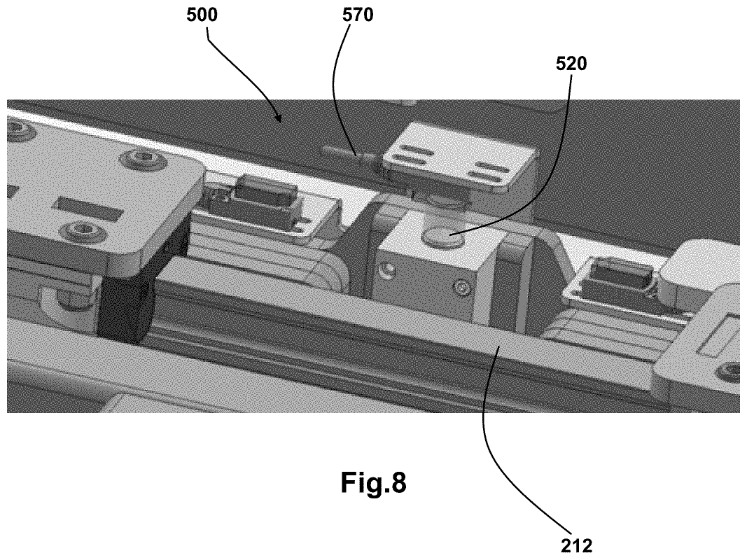

The first rail 211 is suitable for guiding the seat 300 (which is not depicted in FIG. 7) along the axis Z. The second rail 212 is suitable for guiding a carriage 510 of the motion-inducing device 500. In operation, which is to say when the seat 300 is to be moved into the test position, the seat 300 is anchored to the carriage 510 using a locking bolt 520. This locking bolt 520 is more particularly illustrated in FIG. 8.

The frame second part 220, illustrated in FIGS. 1 and 2, extends vertically perpendicular to the frame first part 210, along the axis Z. This frame second part 220 is positioned at one end of the travel of the mobile carriage 300. The frame third part 230 is supported by the frame second part 220. This third part 230 forms a partial cage comprising a vertical base 231 and two triangular components 232A, 232B, with cutouts, extending laterally from the vertical base 231. The third part 230 rests on the frame first part 210. The frame fourth part 240 is supported by the frame third part 230. The frame first part, the frame second part 210, the frame third part 230 and the frame four part 240 are metal components.

The force platform here comprises two parts 400A, 400B making it possible to measure the pushing effort applied by the individual. A first part 400A is suitable for measuring pushing effort on the individual's right side. A second part 400B is suitable for measuring pushing effort on the individual's left side. The force platform 400A, 400B makes it possible to measure the forces generated by the individual on pushing off and/or on landing. This force platform 400A, 400B is referred to as having “six degrees of freedom”. It is thus able to measure forces in three independent directions in space (in the direction perpendicular to the movement, and also in the plane perpendicular to the movement) and three moments leading to rotations about three independent axes. The platform employs sensors of the piezoelectric type suitable for generating a signal having a passband at least equal to 1000 Hz. The platform can be repositioned automatically in the direction of the individual and also in the plane perpendicular to the movement (height and width) so as to ensure optimal quality measurement. These movements are performed by a set of automated systems controlled by the user on the basis of recorded data or on demand.

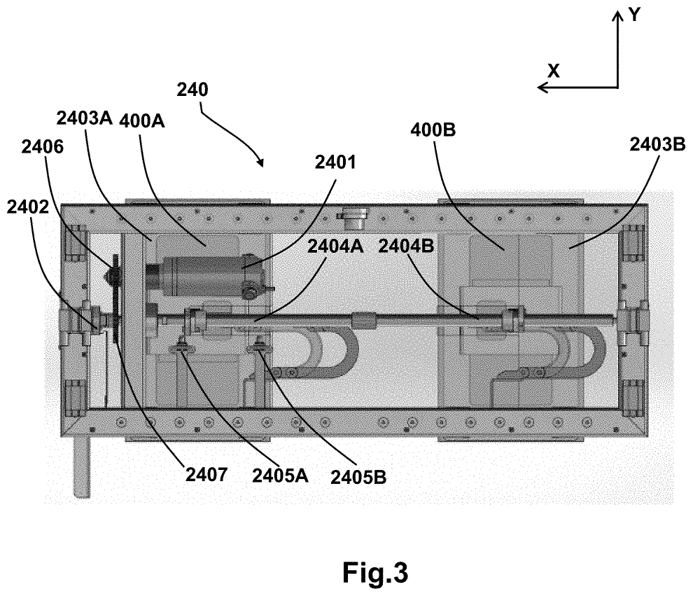

The first part 400A and the second part 400B of the platform are positioned on the frame fourth part 240. The spacing, which is to say the distance in the X direction, between the first part 400A and the second part 400B of the force platform is adjusted by means of a first DC motor 2401 and of a first incremental encoder 2402, which are illustrated in FIG. 3. The first DC motor 2401 is suitable for moving the first part 400A and the second part 400B relative to one another. The first incremental encoder 2402 is suitable for delivering pulses enabling the definition of a direction and of a count. Typically, an incremental encoder comprises a disk having opaque zones and transparent zones. An LED diode emits a ray of light which impinges on photodiodes as each transparent zone of the disk moves past.

In FIG. 3, the force platform also comprises:
a first plate support 2403A and a second plate support 2403B,
a first ball screw 2404A and a second ball screw 2404B,
a first inductive detector 2405A and a second inductive detector 2405B,
a first torque limiter 2406;
a first gearset 2407.

The plate supports 2403A, 2403B are respectively suitable for supporting the first part 400A and the second part 400B of the platform. These plate supports are guided by rails (not visible in FIG. 3) fixed to the frame fourth part 240. Blockers (not visible in FIG. 3) make it possible to limit mechanical lash during tests. The separation of the plate supports 2403A, 2403B is performed symmetrically. The first ball screw 2404A and the second ball screw 2404B have opposite hands to one another and are secured to one another. They are turned by the DC motor 2401 via a first gear set 2407. The first torque limiter 2406 here is a friction-type torque limiter. It guarantees safety during the movements of the plate supports 2403A, 2403B. The first incremental encoder 2402 makes it possible to determine the position of the plate supports 2403A, 2403B. Upon switch on or after an emergency stop, this first incremental encoder 2402 has to be initialized. The “zero” point for the encoder will be determined by separating the plate supports 2403A, 2403B. The two inductive detectors 2405A, 2405B act as software end stops to halt the movement of the plate supports 2403A, 2403B as soon as one of the two inductive detectors 2405A, 2405B is activated. An operator will manually control the separation of the plate supports 2403A, 2403B via a human-machine interface and/or using a wired remote control. This control can be performed during a setup phase for adapting the spacing to suit the build of the individual. This control can also be performed in manual mode or in a maintenance mode.

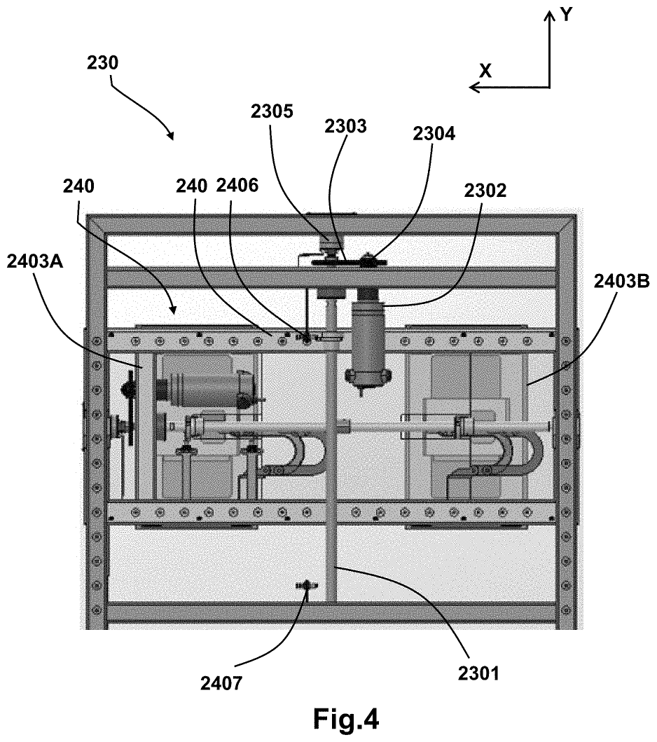

The frame fourth part 240 is mounted with the ability to move on the frame third part 230. As illustrated in FIG. 4, the frame fourth part 240 is moved height wise by means of a ball screw 2301 which is turned by a DC motor 2302 via a gearing system 2303. A friction-type torque limiter 2304 guarantees safety during movement of the frame fourth part 240. Blockers (not depicted) make it possible to limit mechanical lash during tests. An incremental encoder 2305 also makes it possible to determine the position of the plates along the axis Y. Upon switch on or after an emergency stop, this incremental encoder 2305 needs to be initialized. The “zero” point for the encoder is determined by raising the plate supports 2403A, 2403B to their highest position. The two inductive end-of-travel sensors 2406 and 2407 act as software end stops to halt the movement of the plates 2403A, 2403B as soon as one of the two end-of-travel sensors is activated. The operator can control the height of the plates 2403A, 2403B manually via a human-machine interface and/or a wired remote control during a setup phase so as to adapt the height of these plates to suit the build of the patient. The height of these plates may also be adapted in manual mode or in maintenance mode.

The frame third part 230 is suitable for moving on the frame first part 210 so as to adjust the position thereof. The frame third part 230 thus moves along the axis Z by means of a toothed belt driven in rotation by a three phase motor controlled by a variator. A friction-type torque limiter guarantees safety during movement of the frame. Blockers make it possible to limit mechanical lash during tests. An incremental encoder makes it possible to determine the position of the first part 400A and of the second part 400B of the platform along the axis Z. Upon switch on or after an emergency stop, the encoder needs to be initialized. The “zero” point for the encoder is determined by moving the first part 400A and the second part 400B back to their farthest position. Two inductive end-of travel sensors act as software end stops to halt the movement of the first part 400A and of the second part 400B of the platform as soon as one of these end-of-travel sensors is activated. Thus, the frame third part 230 is suitable for adopting an initial position, referred to as the farthest-back position, in which this frame third part 230 is as far back as possible. The blockers block the frame third part 230 in this position. This frame third part 230 is suitable for also adopting an intermediate position that is offset along the axis Z relative to the initial position. Finally, the frame third part 230 is suitable for adopting a final position, referred to as the farthest-forward position, which is offset from the intermediate position along the axis Z.

Once the position adjustment has been made, the force platform 400A, 400B is locked by a pneumatic blocker. This enables the measurements to be repeatable.

In order to take the measurements, the individual needs to be positioned on the seat 300. This seat 300 is adjustable and allows the individual to be placed on it either seated or lying down.

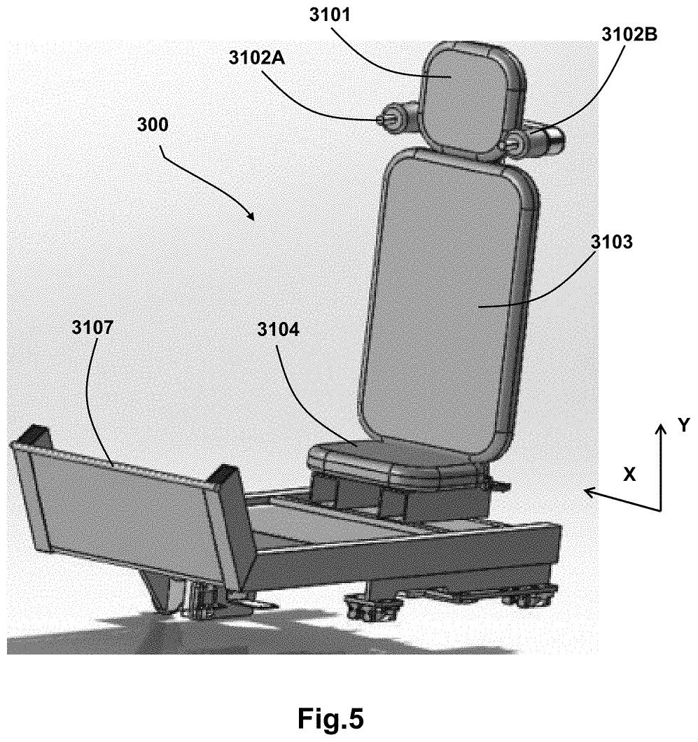

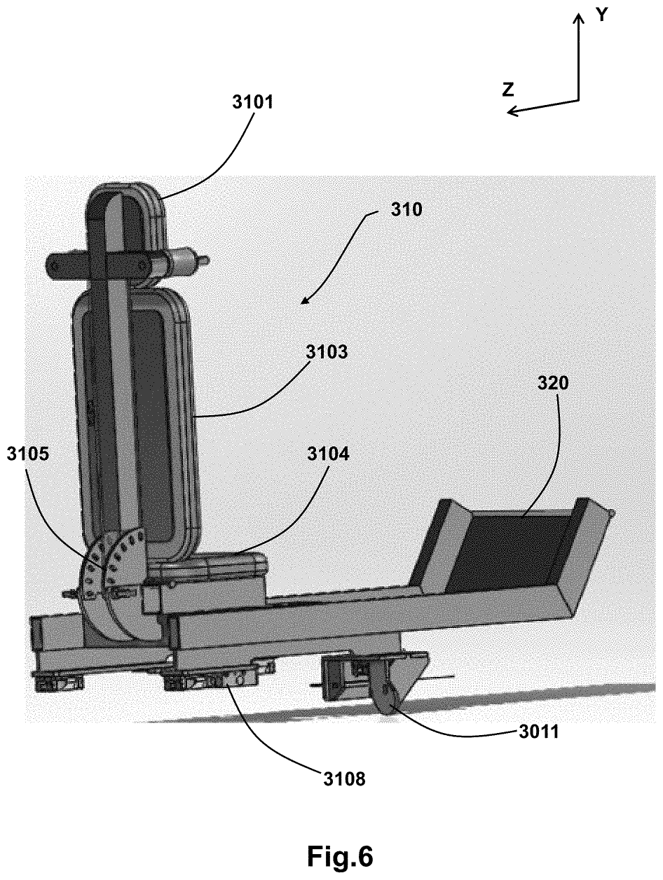

As illustrated in FIG. 5 and in FIG. 6, the seat 300 more particularly comprises:
a head rest 3101;
two handgrips 3102A, 3102B;
an inclinable backrest 3103 that can be inclined via an inclination adjustment device 3105;
a seating portion 3104;
a footrest 3106.

In order to perform the tests, the patient takes up a position on the seat 300. This seat 300 can move freely along the axis Z via the rail 211. Prior to the test, the seat 300 is blocked by a suitable locking/unlocking device 3108 more particularly visible in FIG. 6.

An inductive end-of-travel sensor in an access zone allows the seat 300 to be positioned precisely. Another inductive end-of-travel sensor in the test zone also allows the seat to be positioned precisely. A second incremental encoder (not visible in FIG. 7) makes it possible to determine the position of the seat 300. This second incremental encoder is a wired incremental encoder. Blocking the seat 300 in the predetermined test position makes it possible for the measurements to be repeatable. For safety reasons, the seat 300 is blocked without the use of energy.

It will be noted that the seat 300 in its outermost position has been set up to be able to accommodate individuals with disabilities, particularly wheelchair users.

It will also be noted that the seat 300 comprises a buffer 3011, as illustrated in FIG. 6. This buffer 3011 assists with damping the seat 300 at the end of travel.

It will also be noted that a cable for the “client” encoder used for measurements is fixed to the seat.

The seat 300 is suitable for being moved on the first rail 211 of the frame first part 210 via the motion-inducing device 500, more particularly illustrated in FIG. 7. As has already been specified, this motion-inducing device 500 comprises a carriage 510 and a locking bolt 520. The motion-inducing device 500 also comprises:
a three phase motor 530;
a toothed belt 540;
a position sensor 550 for the position of the carriage 510;
a speed detector 560 detecting the speed of the carriage 510;
a position detector 570 detecting the position of the locking bolt 520.

The carriage 510 is suitable for moving along the second rail 212 via the toothed belt 540. This toothed belt 540 is driven by the three phase motor 530. The position of the carriage 510 is determined using the position sensor 550. This position sensor 550 is a laser position sensor. The speed of the carriage 510 is also determined from a speed detector 560 such as an inductive sensor. In a variant, the speed detector is of the potentiometer type and covers the entire range of movement of the carriage. It is precise for example to within the order of 1 millimeter. The speed of the carriage 510 can be varied according to the distance separating it from the seat 300, when the latter is immobilized in the test phase. The speed of the carriage 510 is controlled by a variator. The variator allows the carriage 510 to move quickly when it is not anchored to the seat 300. The variator also limits the speed of the carriage 510 when this carriage is very close to the seat 300. The movement of the carriage 510 is then stopped when it is positioned in alignment with the seat 300. The carriage 510 is anchored to the seat 300 using the locking bolt 520. This locking bolt 520 is able to adopt a deployed position in which the locking bolt 520 mechanically connects the carriage 510 to the seat 300 and a retracted position in which the locking bolt 520 does not anchor the carriage 510 to the seat 300. The position of the locking bolt is detected by the position detector 570. This position detector 570 is, for example, a detector of inductive type.

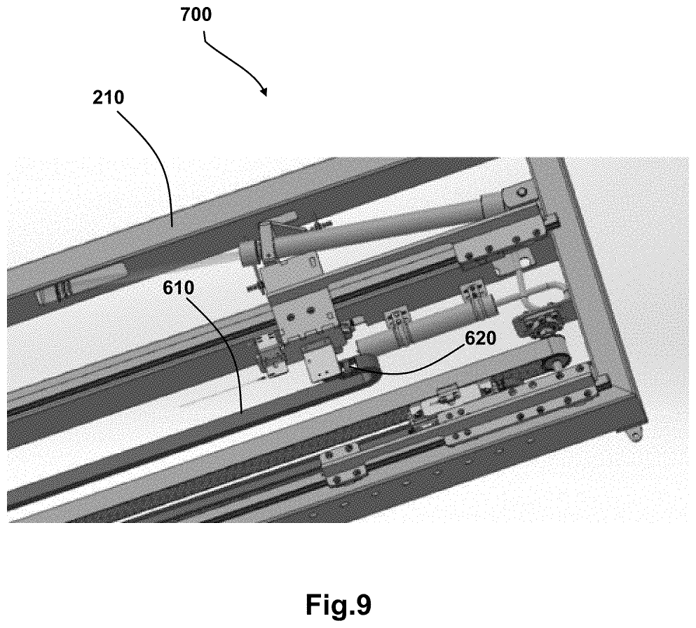

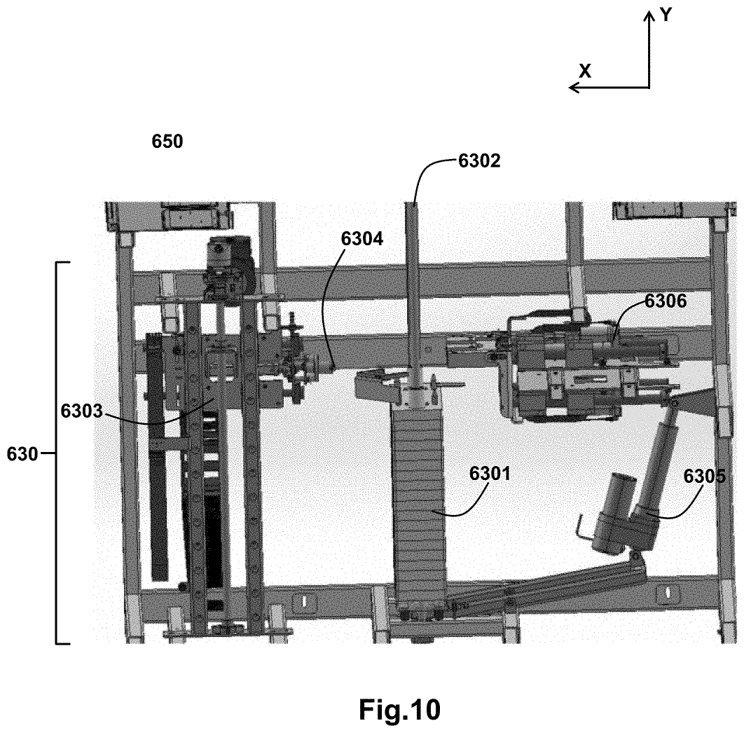

In order to take measurements on the individual, it is necessary to load the seat 300. This loading of the seat is performed using a loading device 600 illustrated in FIGS. 9, 10 and 11. Such a device 600 comprises:
a strap 610;
a lock 620;
a mobile-mass management system 630.

As is illustrated in FIG. 9, the strap 610 is positioned between a lock 620 positioned beneath the seat 300 and the mobile-mass management system 630. This management system is suitable for automatically generating a load with a view to tensioning the strap 610 and consequently prestressing the seat 300. This load is variable and is dependent on the conditions under which the individual is to be stressed. The load may thus adopt a value of between 0 kg and 207.5 kg, in increments of 2.5 kg. The mobile-mass management system 630 thus comprises:
a stack 6301 comprising a plurality of weights;
a bar 6302;
an indexer 6303;
a pin 6304 held in the indexer 6303 by an electromagnet and intended to be inserted into the stack 6301;
a lifting device 6305 for lifting the stack 6301;
an additional loading device 6306.

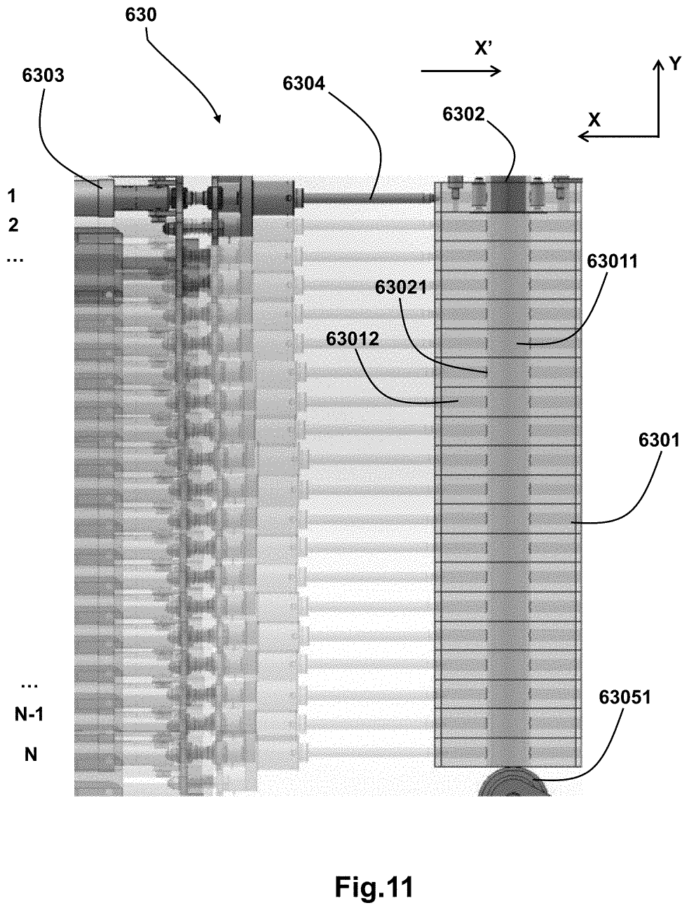

FIG. 11 more specifically illustrates the operation of the mobile-mass management system 630. The stack 6301 is lifted using the lifting device 6305. In FIG. 11, just part of a roller 63051 is depicted. This roller 63051 supports the stack 6301 via the lowermost weight of said stack. The indexer 6303 is suitable for moving along the axis Y in order to adopt N positions, N being dependent on the number of weights making up the stack 6301. Each weight has a vertical passage 63011 extending in the direction Y and a horizontal passage 63012 extending along the axis X. The vertical passages 63011 of the various weights form a channel in which the bar 6302 is able to slide. This bar 6302 also comprises a plurality of horizontal passages 63021 which are coaxial with the horizontal passages 63012 in the various weights.

The indexer 6303 is positioned along the axis Y relative to the stack 6301 according to the load selected. Once the indexer 6303 is in position, the pin 6304 is moved by the indexer 6303 in the direction X′ toward the weight facing it. Through this movement, the pin 6304 will enter the horizontal passage 63012 of the weight and pass through the bar 6302 at the horizontal passage 63021. The pin 6304 is then released from the indexer 6303 and the bar 6302 is thus mechanically connected to all of the weights of the stack 6301 that are positioned above the pin 6304. This collection of weights constitutes the predetermined load that will be applied to the seat 300 of the measurement bench 100 via the strap 610. It will be noted that the weights which are situated below the pin 6304 are not connected to the bar 6302, which slides within them when the lifting device 6305 lowers the stack 6301.

It is possible to add additional weights to the stack 6301 using the additional loading device 6306. A load of 5 kg and/or a load of 2.5 kg may therefore be added, for example in the form of two 1.25 kg weights. These additional weights are positioned by an actuating cylinder of the device 6306 on the weight at the top of the stack 6301.

As illustrated in FIG. 9, the damping device 700 comprises an actuating cylinder 710 comprising:
an actuating cylinder body 7101;
an actuating cylinder rod 7102;
a reservoir 7103.

The damper rod 7101 is suitable for damping the seat 300 following a phase in which the individual applies an impulse. It is the buffer 3011 of the seat 300 that will come into contact with the rod 7101 in order to halt the seat 300. In one particular embodiment, the damper 710 is of the oleo pneumatic type and a fluid circulates between the damper body 7101 and the reservoir 7103. The damping device 700 makes it possible to brake the seat assembly in complete safety at the end of its travel. Thereafter, the motion-inducting device 500 returns the seat 300 to the test position at reduced speed. The damping device thus makes it possible to measure the movements of multiple joints in complete safety and without apprehensiveness on the part of the individual.

The measurement bench 100 also comprises a signal acquisition device. This device makes it possible to digitize signals from sensors, such as the piezoelectric sensors of the force platform 400A, 400B or the speed sensor 560 and/or the position sensor 570 of the carriage 510. The digitization frequency is programmable. For example, this frequency is set at 1000 Hz. The data from the various sensors are acquired synchronously. The signal acquisition device is equipped with a synchronization input so that auxiliary devices such as a surface electromyograph, known by the abbreviation EMG, or segment positioners can be used. This synchronization input thus makes it possible to take specific measurements. Acquisition software makes it possible to preprocess the raw data so as to obtain force/speed profiles. This acquisition software thus has the function of logging the raw data regarding the measurement in a specified format with a keyword description. It allows the user to check the relevance of the preprocessed raw data and validate the current measurement. The acquisition software interacts via a computer network with the controller that manages the functions of the seat so as to command the positioning operations and log the parameters. The data are logged anonymously by the digital indicator. The date-stamping of the measurements and the incrementation of the logged data are automatic.

Processing software that performs the processing afterward allows the results to be interpreted and exploited.

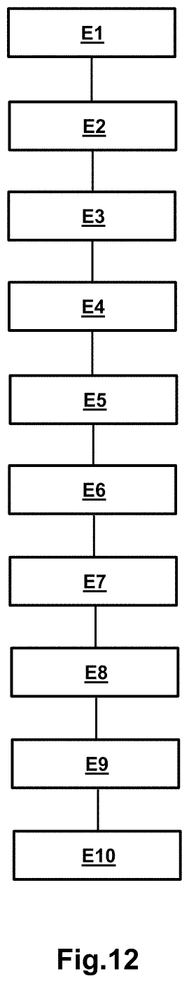

FIG. 12 illustrates the steps of a characterization method for neuromuscular characterization of at least one limb of the individual using the measurement bench 100. This method comprises a first step E1 in which the individual is positioned on the seat 300 of the measurement bench 100 with assistance from an operator in the access zone. When the patient is correctly positioned, the operator informs a central system via the human-machine interface that the individual can be brought into the test position. The carriage 510 is anchored to the seat 300 by the locking bolt 520 and brings the patient into the test position in a step E2. This test position is a position that is farther back than a standard position in which the individual would be in contact with the force platform via the limb or limbs that the operator is seeking to stress. The seat 300 is locked in the test position by the locking/unlocking device 3108 in a step E3. The carriage 510 is unanchored from the seat 300 and returns to a parking zone. The damping device 700 takes up its position. It will also be noted that the seat 300 is designed to limit resistive effort as far as possible and to have minimal inertia.

At this stage, there are two possible scenarios: either the morphological information regarding the individual is known or it is not known or needs to be modified. In the first instance, the plate supports 2403A, 2403B will move on the frame 200 to attain the known positions under the authorization of the operator. When the positions are attained, the operator confirms that they are compliant. In the second instance, the operator moves the plate supports 2403A, 2403B to position them suitably on the frame 200. When the positions of the plate supports 2403A, 2403B are compliant, the operator validates them on the human-machine interface and the machine informs a “client” computer system that it is ready and makes the morphological data available. A communication of the “web server” type allows exchanges between this human-machine interface and this “client” computer system.

Before each test, the “client” computer system informs the loading device 600 of the predetermined load to which the seat 300 is to be subjected in a step E4. If this load is zero, the strap 610 is unanchored from the seat 300. If not, the load can vary from 10 kg to 207.5 kg in increments of 2.5 kg and the strap 610 is anchored to the seat 300. When the predetermined load is in position, the machine informs the “client” computer system that the test can begin. In order not to tire the individual, the seat remains locked during the loading step E4. When the “client” computer system authorizes the test and the seat 300 is released in a step E5. The individual is then driven toward the force platform 400A, 400B by the predetermined load. In the test position, the individual does not touch the force platform. The distance separating the individual from this platform, combined with the predetermined load, thus makes it possible to generate an acceleration upon the unlocking of the seat 300.

In a step E6, the individual lands on the force platform 400A, 400B. This individual can then, in a step E7, propel themselves away from this force platform 400A, 400B, using their legs or their arms. In a step E8, signals are acquired. These signals concern the force generated by the individual on the force platform 400A, 400B during the landing step E6 and/or the push off step E7. Following the individual's push off step E7, the seat 300 is damped in a damping step E9 by the damping device 700. Once halted, the seat 300 is blocked and once again anchored to the mobile carriage 510. The individual is then returned at a slow speed either to the test position for a further test or to the access zone so as to allow this individual to leave the measurement bench 100. The measurement bench 100 is then ready to receive another individual.

It will be noted that the step E8 of acquiring the signals involves the digitizing of said signals.

It will also be noted that the data processing step E10 is performed afterward.

The characterization method that forms the subject of the invention and the associated measurement bench 100 afford the following advantages:
of precisely measuring the force-speed profiles and explosive activity of the lower and upper limbs with a view to characterizing the individual or the physical performance;
of measuring muscle and functional imbalances for prophylactic and medical purposes;
of taking these measurements in complete safety with no prior learning, and on any individual, whether sporting, non-sporting, or in the process of rehabilitation; of measuring the deformation of the profile as a result of eccentric effort similar to landing or to descending a staircase;
of measuring the effect of fatigue on the force-speed profile, for prophylactic or performance-related purposes;
of taking measurements under dynamic conditions;
of measuring the deformation of the profile following a concentric effort similar to a jump of the “drop jump” type;
of measuring a human movement in ecological fashion, which is to say within the meaning of a real movement.

The invention is not restricted to the embodiments and variants set out and other embodiments and variants will be clearly apparent to the person skilled in the art.

Thus, the measurement bench also makes it possible to take static measurements when the test position coincides with the standard position. In such instances, the individual is in contact with the force platform 400A, 400B right from the start of the test.

Thus, the measurement bench can be used for musculoskeletal and osteo-articular applications, in rehabilitation or in return-to-sport.

Thus, the measurement bench may use, for the force platform, technologies other than piezoelectric sensors.

1. A characterization method for the neuromuscular characterization of at least one lower and/or upper limb of an individual using a measurement bench, said characterization method comprising:
a step (E1) of placing the individual on a seat of the measurement bench;
a step (E2) of moving the seat into a test position, said test position being farther back than a standard position wherein the individual is in contact with a force platform via the at least one limb of the individual, said force platform being intended to measure a force generated by said at least one limb of the individual;
a step (E3) of locking the seat in the test position;
a step (E4) of loading the seat with a predetermined load;
a step (E5) of unlocking the seat, said seat being driven toward the force platform by the predetermined load;
a step (E6) wherein the individual lands on the force platform;
a step (E7) wherein the individual pushes against the force platform;
a step (E8) of acquiring signals regarding the force generated by the individual on said force platform during the landing step (E6) and/or the pushing step (E7);
a step (E10) of processing data from said acquired signals.

2. The characterization method as claimed in claim 1, wherein said characterization method comprises a step (E9) of damping the seat after the step (E7) of pushing by the individual.

3. The characterization method as claimed in claim 1, wherein the seat is returned, after the step (E7) of pushing by the individual, to the test position or to a removal position for removing the individual from the measurement bench.

4. The characterization method as claimed in claim 1, wherein the speed of movement of the seat toward the test position and/or toward the removal position is controlled.

5. The characterization method as claimed in claim 4, wherein the speed of movement of the seat is controlled by a carriage anchored to said seat.

6. The characterization method as claimed in claim 5, wherein the mobile carriage is unanchored from the seat when said seat is locked in the test position.

7. The characterization method as claimed in claim 1, wherein, prior to the step (E5) of unlocking the seat, the position of the force platform is adjusted on part of a frame of the measurement bench to suit the individual.

8. The characterization method as claimed in claim 1, wherein the step (E8) of acquiring the signals involves the digitizing of said signals.

9. The characterization method as claimed in claim 1, wherein the data processing step (E10) is performed afterward.

10. A measurement bench for the neuromuscular characterization of at least one lower and/or upper limb of an individual using the characterization method as claimed in claim 1, said measurement bench comprising:
a frame intended to rest on the floor;
a force platform intended to measure a force generated by the at least one limb of the individual;
a seat intended to receive the individual, said seat being designed to be able to move with respect to the frame as far as a test position, said test position being farther back than a standard position wherein the individual is in contact with the force platform via the at least one limb of the individual;
a locking/unlocking device suitable for locking/unlocking the seat in the test position, said seat being driven toward the force platform by a predetermined load upon unlocking;
a loading device for loading the seat with the predetermined load;
an acquisition device acquiring signals regarding the force generated by the individual on the force platform when said individual lands on and/or pushes against said force platform, said acquisition device comprising signal acquisition software;
processing software for processing data from the acquired signals.

11. The measurement bench as claimed in claim 10, wherein the force platform is suitable for measuring said force generated by the individual in three mutually perpendicular directions and for measuring three moments about said perpendicular directions.

12. The measurement bench as claimed in claim 10, wherein the force platform is in at least two parts.

13. The measurement bench as claimed in claim 10, wherein the force platform comprises sensors of the piezoelectric type suitable for generating a signal having a passband at least equal to 1000 Hz.

14. The measurement bench as claimed in claim 10, wherein said measurement bench comprises a carriage and a locking bolt, said locking bolt being able to adopt a deployed position wherein the locking bolt mechanically connects the carriage to the seat and a retracted position wherein the locking bolt does not connect said carriage to said seat.

15. The measurement bench as claimed in claim 14, wherein said measurement bench comprises a toothed belt suitable for moving the carriage and a three phase motor suitable for actuating said toothed belt, the speed of movement of the carriage being controlled by a speed sensor.

16. The measurement bench as claimed in claim 10, wherein the loading device comprises an indexer and a stack of a plurality of weights, said indexer being suitable for inserting a pin into the stack so as to select the predetermined load.

17. The measurement bench as claimed in claim 10, wherein said measurement bench comprises a damping device to damp the seat after a push from the individual.

18. The measurement bench as claimed in claim 10, wherein the processing software operates afterward.
